(19) United States
(12) Patent Application Publication
(10) Pub. No.: US 2018/0258659 A1
(43) Pub. Date: Sep. 13, 2018

(54) BUNKER SYSTEM FOR RADIATION THERAPY EQUIPMENT

(71) Applicant: NEW ENGLAND LEAD BURNING COMPANY, INC., Burlington, MA (US)

(72) Inventors: Richard J. LeBlanc, Newton, MA (US); Gary J. Miller, Saugus, MA (US)

(21) Appl. No.: 15/914,357

(22) Filed: Mar. 7, 2018

Related U.S. Application Data
(60) Provisional application No. 62/467,866, filed on Mar. 7, 2017.

(51) Int. Cl. E04H 3/08; E04B 2/08; E04B 2/84; E04B 5/32; E04B 7/20; E02D 27/44; E04C 1/00; E06B 5/18; E04C 5/06; A61N 5/10

(52) U.S. Cl. CPC E04H 3/08; E04B 2/08; E04B 2/84; E04B 5/32; E04B 7/20; E04B 2002/021; E04C 1/00; E06B 5/18; E04C 5/06; A61N 5/10; E02D 27/44

(57) ABSTRACT

A bunker system for shielding radiation emitted from a radiation treatment device includes a multi-core wall structure that completely surrounds the radiation treatment device. The wall structure includes a cast-in-place concrete inner core of limited thickness in order to minimize curing time requirements. The inner core is immediately surrounded by an outer core constructed from a plurality of preformed modular blocks. Each modular block is constructed of a radiation shielding material, such as concrete. As part of the assembly process, the preformed modular blocks are designed to be stacked top-to-bottom and side-by-side in an interlocking fashion to form a continuous wall structure, with blocks additionally arranged in a front-to-back relationship to achieve the required outer core thickness. The dual-core construction of the wall structure enables the bunker system to be quickly and efficiently assembled with enhanced quality control and potential reusability.

### FIELD OF THE INVENTION

The present invention relates generally to radiation therapy equipment and, more particularly, to bunker systems designed to shield radiation generated from such equipment.

### BACKGROUND OF THE INVENTION

In the medical field, radiation is commonly utilized to treat cancer by emitting focused, high-energy waves onto a patient in order to shrink and/or destroy tumor cells without significantly impinging healthy cells. Radiation is generated and delivered using radiation therapy equipment located at designated treatment facilities, such as hospitals and cancer treatment centers.

To prevent the escape of radiation outside of the designated treatment area, radiation therapy equipment is traditionally located within an enlarged bunker system, or radiation room. Due to its critical importance in effectively shielding radiation, a bunker system is typically engineered by a physicist with expertise in the field as part of the architectural process in designing a radiation treatment facility. Notably, the physicist provides a detailed physics design of the bunker system which specifies the radiation shielding requirements that will ensure that radiation does not escape the bunker system.

Traditionally, bunker systems include a plurality of thick concrete walls which are arranged in a particular configuration so as to completely surround the radiation generating equipment. As can be appreciated, thick concrete walls not only provide long-term durability and structural support but, most importantly, serve as highly effective radiation shielding elements.

The concrete walls of a conventional radiation bunker system are generally formed using a cast-in-place construction process. Specifically, each wall is constructed by depositing a concrete mixture, while in an unhardened state, between enlarged, removable forms and, in turn, allowing the mixture to harden during a specified curing period. In radiation shielding applications, cast-in-place concrete walls are generally substantial in thickness (e.g., 3-10 feet thick) to meet requisite radiation shielding standards.

Traditional bunker systems constructed in the manner as set forth above have been found to experience certain notable drawbacks.

As a first drawback, bunker systems of the type as described above are constructed in a time-consuming fashion due, largely in part, to the inclusion of cast-in-place concrete walls of considerable thickness. Most notably, the curing period for concrete walls of such thickness often reaches or exceeds several months in duration. As a result, full operability of a radiation therapy room is often not achieved until at least one year after initiating construction.

As second drawback, bunker systems of the type as described above are commonly constructed in a largely inefficient fashion, which may cause an increase in overall labor costs. Specifically, prior to the deposition of cast-in-place concrete within the removable forms, preparatory work associated with the installation of radiation thereby equipment needs to be completed. Notably, conduits for electrical wiring, ductwork for the delivery and exhaust of gasses and coolants, as well as any other required installation infrastructure located within and through the concrete walls needs to be positioned in its entirety prior to the deposition of the unhardened concrete. Only after such work has been completed can concrete be deposited within the formwork to construct a traditional, cast-in-place bunker assembly. Furthermore, only after the concrete walls have fully cured can certain final installation processes be undertaken (e.g. mechanically securing equipment to the concrete, internal wall framing and finishes, as well as other moisture sensitive activities). This inability to enable different workers to engage in the various installation steps in parallel, coupled with the significant cure time associated with the concrete walls, creates significant delays in the overall construction process.

As a third drawback, bunker systems of the type as described above are often constructed without adequate quality control. In particular, due to the considerable thickness required for each wall, cast-in-place concrete is often deposited through a multi-stepped pouring process that can create notable variances in density within each wall (e.g., due to the inadvertent introduction of air therein or the presence of voids, cold joints, and honeycombs). As can be appreciated, such variances in concrete density can compromise the shielding performance of certain walls, which is highly undesirable.

### SUMMARY OF THE INVENTION

It is an object of the invention to provide a new and improved bunker system for radiation therapy equipment.

It is another object of the present invention to provide a bunker system as described above that is configured to completely surround the radiation therapy equipment and effectively shield the outside environment form any radiation generated therefrom.

It is yet another object of the present invention to provide a bunker system as described above that can be constructed and fully operable within a limited duration of time.

It is still another object of the present invention to provide a bunker system as described above that readily allows for the installation of any required radiation therapy equipment infrastructure, such as ductwork, wiring and the like, during its construction process.

It is yet still another object of the present invention to provide a bunker system that maintains reliable, consistent and uniform radiation shielding characteristics.

Accordingly, as a feature of the present invention, there is provided a bunker system for radiation therapy equipment, the bunker system comprising (a) an inner core, and (b) an outer core that immediately surrounds the inner core, (c) wherein the inner core and outer core together form a radiation shielding structure.

Various other features and advantages will appear from the description to follow. In the description, reference is made to the accompanying drawings which form a part thereof, and in which is shown by way of illustration, an embodiment for practicing the invention. The embodiment will be described in sufficient detail to enable those skilled in the art to practice the invention, and it is to be understood that other embodiments may be utilized and that structural changes may be made without departing from the scope of the invention. The following detailed description is therefore, not to be taken in a limiting sense, and the scope of the present invention is best defined by the appended claims.

### BRIEF DESCRIPTION OF THE DRAWINGS

In the drawings wherein like reference numerals represent like parts:

FIG. 1 is a longitudinal section view of a novel bunker system for radiation therapy equipment, the bunker system being constructed according to the teachings of the present invention;

FIG. 2 is top section view of the bunker system shown in FIG. 1, taken along lines 2-2;

FIG. 3 is a top perspective view of the bunker system shown in FIG. 2, taken along lines 3-3, the bunker system being shown in a partially assembled state to illustrate novel aspects of the present invention;

FIG. 4 is a top plan view of the bunker system shown in FIG. 1;

FIGS. 5(a) and 5(b) are front and top plan views, respectively, of one of the modular blocks used to form the outer core in FIG. 1; and

FIG. 5(c) is a section view of the modular block shown in FIG. 5(b), taken along lines 5C-5C.

### DETAILED DESCRIPTION OF THE INVENTION

### Bunker System 11

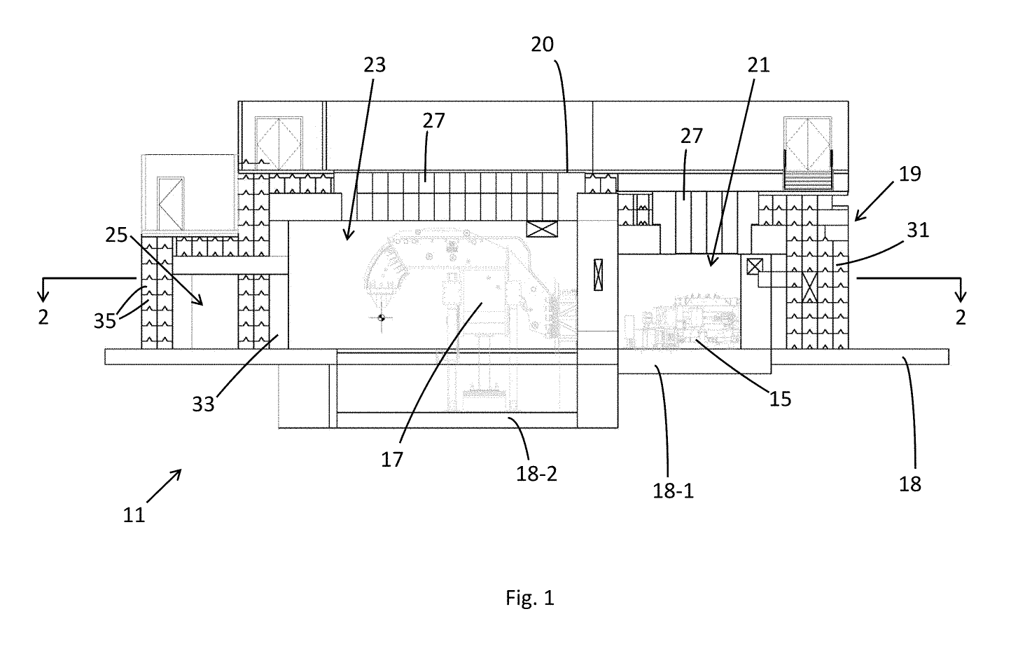

Referring now to FIGS. 1-3, there is shown a novel bunker system constructed according to the teachings of the present invention, the bunker system being identified generally by reference numeral 11. As will be explained in detail below, bunker system 11 utilizes a multi-core radiation shielding construction which provides several notable advantages including, but not limited to, quick and efficient assembly, enhanced quality control, and reusability.

In the description that follows, bunker system 11 is explained in the context of providing radiation shielding for a radiation treatment device 13. As defined herein, radiation treatment device 13 represents any instrument in the radiation therapy field that is able to deliver focused, high-energy waves to a patient. Accordingly, it should be understood that the bunker system 11 is not limited for use in connection with any particular type or style of radiation therapy machine. Rather, bunker system 11 could be modified, as deemed necessary, to provide radiation shielding for alternative types of radiation treatment devices without departing from the spirit of the present invention.

As represented herein, radiation treatment device 13 comprises a cyclotron 15 that supplies radiation to a radiation delivery apparatus 17 which is located in a different region of bunker system 11. Radiation delivery apparatus 17, represented herein as a gantry, allows for high-energy radiation to be directed onto a patient at various angles along a circular path.

As seen most clearly in FIG. 1, bunker system 11 comprises (i) a concrete-based foundation, or flooring structure, 18, (ii) a wall-type radiation shielding structure 19, and (c) a ceiling structure 20. As can be appreciated, the particular construction of wall-type shielding structure 19 provides bunker system 11 with a number of notable advantages and, as such, serves as the principal novel feature of the present invention.

In the present embodiment, bunker system 11 is shown in its entirety as standalone facility. However, it should be noted that bunker system 11 is not limited to the aforementioned construction. Rather, it is to be understood that the principles of the present invention could be applied into alternative building structures without departing from the spirit of the present invention. For instance, the novel aspects of bunker system 11 could be readily incorporated into a large-scale healthcare facility, such as a hospital, as a designated region, or floor, thereof.

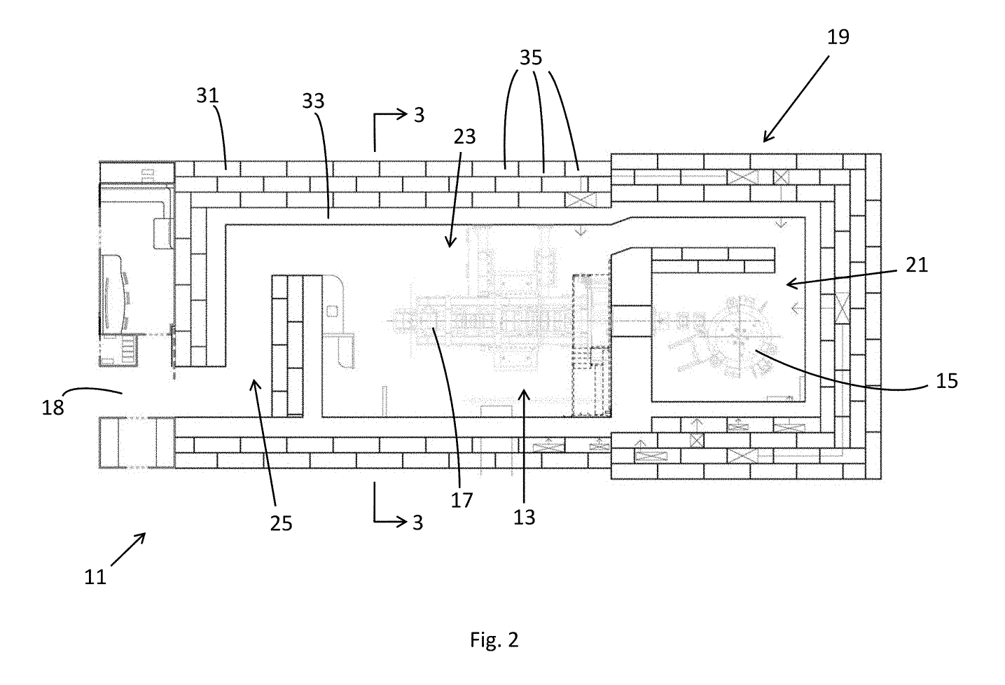

As seen most clearly in FIGS. 1 and 2, foundation 18, wall structure 19 and ceiling structure 20 together define (i) a substantially enclosed cyclotron vault 21 that is dimensioned to receive cyclotron 15, (ii) a proton treatment room 23 that is dimensioned to receive gantry 17, controls and other machine-related items, and (iii) a maze 25 which serves as the primary means of entrance to and egress from room 23. As the principal function of bunker system 11, the configuration and concrete-based construction of flooring, wall and ceiling structures 18, 19 and 20 ensures that radiation treatment device 13 is surrounded on all sides by a layer of concrete of sufficient thickness to prevent emission of radiation to the outside environment.

It should be noted that the particular dimensions and configuration of the various rooms within bunker system 11 are provided for illustrative purposes only. Preferably, the specific design of bunker system 11 (and, in particular, shielding structure 19) would be customized for a designated radiation treatment facility based on, inter alia, the architectural footprint of the facility as well the particular type of radiation treatment device 13 to be utilized therein. Accordingly, it is to be understood that the configuration of bunker system 11 could be redesigned and optimized for use in alternative facilities without departing from the spirit of the present invention.

For instance, in place of maze 25, shielding structure 19 for bunker system 11 could be provided with direct shielding doors (e.g., bi-parting shielding doors) to afford greater ease of access into proton treatment room 23.

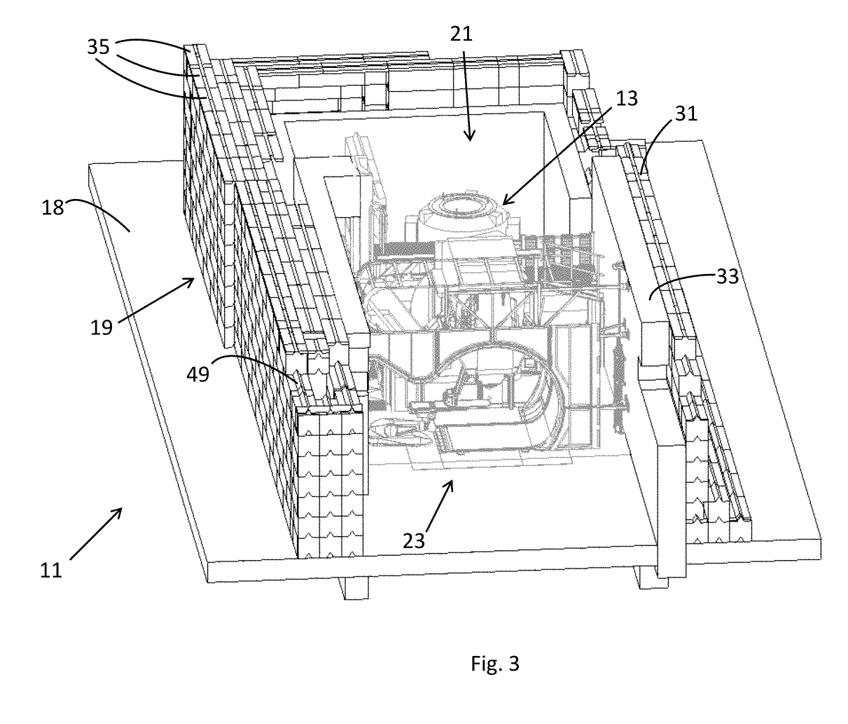

As seen most clearly in FIGS. 1 and 3, foundation 18 is represented herein as a concrete slab of a sufficient thickness to provide (i) suitable radiation shielding capabilities and (ii) structural, load-bearing support for not only the remainder of bunker system 11 but also at least a portion of the overall healthcare facility. Foundation 18 is shown herein as comprising a reinforced region 18-1 directly beneath cyclotron 15 and a sub-floor region 18-2 directly beneath gantry 17. However, it is to be understood that the details of foundation 18 are provided for purposes of illustration only. As a result, the particular design of foundation 18 could be modified, as needed, to suit the structural needs of the designated treatment facility.

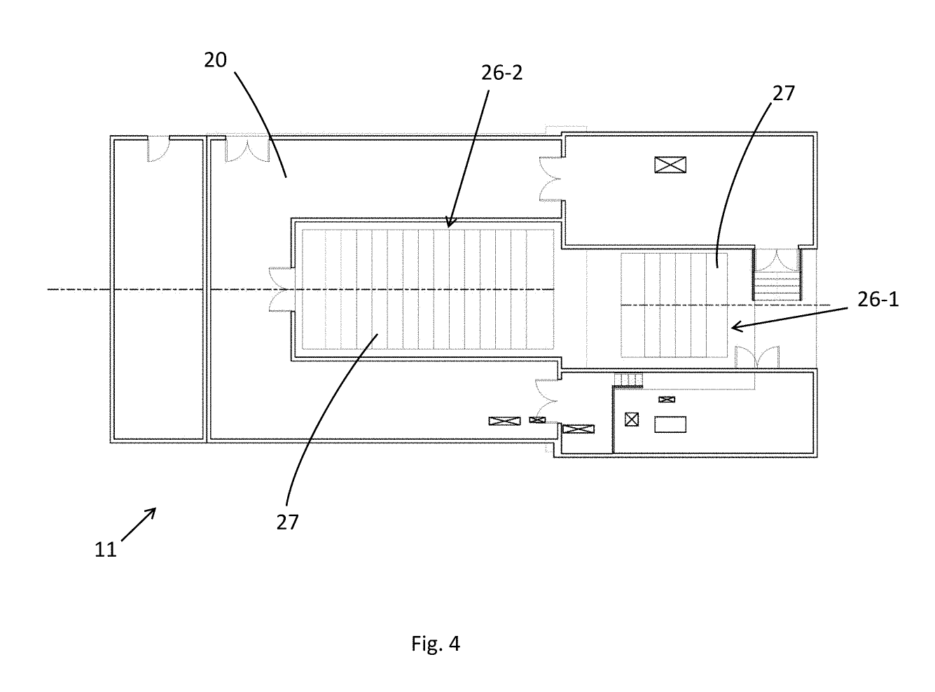

As seen most clearly in FIGS. 1 and 4, ceiling structure 20 is represented herein as a concrete slab of sufficient radiation shielding thickness that is shaped to define enlarged access openings 26-1 and 26-2 through which cyclotron 15 and gantry 17 are delivered into vault 21 and room 23, respectively, during the assembly of bunker system 11. As will be explained further below, ceiling structure 20 additionally includes a plurality of removable concrete panels 27 which together serve to selectively enclose each of the enlarged access openings 26. As can be appreciated, the limited size of each panel 27 facilitates its installation within and/or removal from access openings 26.

### Radiation Shielding Structure 19

As referenced briefly above, the unique construction of radiation shielding structure, or wall, 19 yields a number of notable advantages in connection with the overall assembly and performance of bunker system 11. More specifically, wall-type radiation shielding structure 19 utilizes a dual-core construction that greatly streamlines the assembly process and thereby accelerates the timeline for facility operability, which is a principal object of the present invention.

As seen most clearly in FIGS. 2 and 3, shielding structure 19 comprises an outer wall 31 that immediately surrounds a corresponding inner wall 33. The unique construction of outer wall 31 and its incorporation into shielding structure 19 provides a number of notable advantages and, as such, serves a principal novel feature of the present invention.

Outer wall, or core, 31 comprises a plurality of individual, interlocking, modular blocks 35, each of which is constructed out of a suitable radiation shielding material, such as a concrete-based mixture. As seen most clearly in FIG. 3, modular blocks 35 can be stacked top-to-bottom, side-by-side and/or front-to-back so as to form a relatively thick, continuous shielding wall structure. As will be explained below, any spacing between adjacent blocks 35 is preferably filled with cast-in-place concrete to eliminate the presence of any voids within outer core 31.

Modular block 35 represents any modular, stackable, radiation-shielding block. For instance, each modular block 35 may be selected from the MEGASHIELD™ line of modular concrete blocks that are currently manufactured and sold by NELCO Worldwide of Burlington, Mass. For use in a bunker system configuration of the type shown in FIG. 1, it is preferred that each modular block 35 be both relatively large in size and constructed of a relatively high density material to meet the necessary structural and shielding requirements.

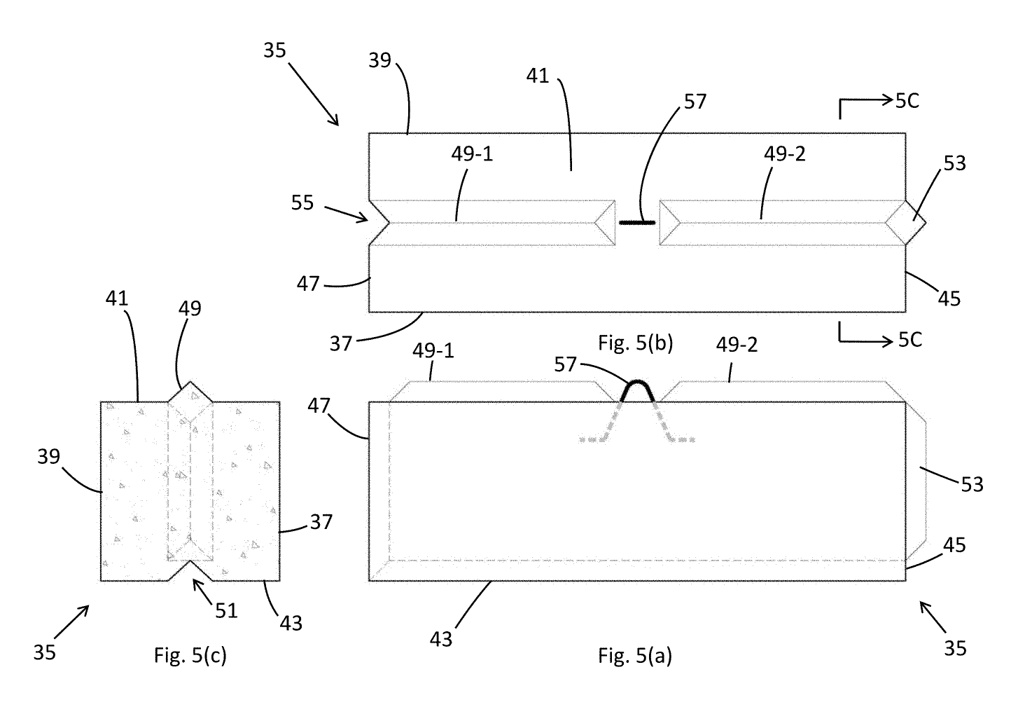

Referring now to FIGS. 5(a)-(c), there is shown an example of a modular block 35 that may be used in the construction of outer wall 31. It should be noted that the details of modular block 35 are provided for illustrative purposes only. Accordingly, it is to be understood that other types of modular radiation shielding blocks could be used in place thereof without departing from the spirit of the present invention.

As can be seen, modular block 35 is a solid, unitary block, generally rectangular in longitudinal cross-section, which is preformed using a concrete-based mixture. Block 35 is shaped to include a flat front surface 37, a flat rear surface 39, a generally flat top surface 41, a generally flat bottom surface 43, and first and second end surfaces 45 and 47.

To assist in the stacking of multiple blocks 35 together so as to form a continuous wall structure, each block 35 is provided with complementary interlocking features. Specifically, top surface 41 is provided with a pair of spaced apart, collinear projections 49-1 and 49-2, each projection 49 being generally triangular in transverse cross-section and protruding outwardly from top surface 45 along its approximate centerline for a portion of its length. In turn, bottom surface 43 is shaped to define a corresponding recess 51, which is generally triangular in transverse cross-section and extending along its approximate centerline for the entirety of its length. As can be appreciated, with a pair of modular blocks 35 stacked one on top of the other, as shown in FIG. 3, recess 51 in the upper block 35 is dimensioned to fittingly receive the pair of projections 49 from the lower block 35, thereby creating an interlocking and properly aligned wall assembly.

In a similar fashion, first, or right, end surface 45 is provided with a projection 53, which is generally triangular in transverse cross-section and protrudes outwardly from its approximate centerline along the majority of its length. In turn, second, or left, end surface 47 is shaped to define a corresponding recess 55, which is generally triangular in transverse cross-section and extends along its approximate centerline for the entirety of its length. As can be appreciated, with a pair of modular blocks 35 arranged in an end-to-end relationship, as shown in FIG. 3, recess 55 in a first block 35 is dimensioned to fittingly receive projection 53 from an adjacent block 35, thereby creating an interlocking and properly aligned wall assembly.

Additionally, modular block 35 is provided with a hook 57 in top surface 41 to facilitate transport. Hook 57 is constructed from a length of a strong and durable material, such as a wire rope, which is formed into an inverted U-shaped configuration with its ends fixedly embedded into top surface 41 between projections 49-1 and 49-2. The exposed central portion of hook 57 extends above top surface 41 and lies coplanar with, or slightly beneath, the tip of projections 49 so as not to interfere with top-to-bottom stacking of blocks 35. As such, using hook 57, a crane or other similar machine can transport each modular block 35 into its desired stacked position within outer core 31 during assembly of shielding structure 19.

As will be explained further in detail below, the use of modular blocks 35 in the construction of radiation shielding structure 19 provides certain notable and, as of yet, unforeseen advantages. Notably, modular blocks 35 provide structure 19 with (i) enhanced quality control, since the density of each block is highly consistent and can be confirmed, as such, through testing, (ii) ease and efficiency in constructing system 11, as machine equipment infrastructure (e.g., electrical wiring and plumbing) can be installed in parallel with the assembly of outer core 31, and (iii) recyclability, since the majority of blocks 35 that form outer core 31 do not experience radioactive contamination and, as a result, can be reused in other bunker systems.

Referring back to FIGS. 1-3, inner wall, or core, 33 is disposed directly inside certain regions of outer core 31 in direct abutment therewith. As seen most clearly in FIG. 2, inner wall 33 surrounds both cyclotron 15 and gantry 17 on all sides, thereby containing any radiation generated therefrom.

Inner wall 33 is preferably constructed using a pour-in-place concrete mixture or any other suitable radiation shielding material. However, due to the presence of outer wall 31, inner wall 33 can be constructed with a thickness that is significantly less than the thickness of typical cast-in-place concrete walls used in traditional radiation shielding environments.

Specifically, inner wall 33 preferably has a thickness of approximately 2 feet, whereas traditional cast-in-place concrete walls often have a thickness in the order of 8-10 feet. As a result, inner wall 33 requires a considerable shorter curing time than traditional cast-in-place concrete walls, thereby accelerating construction of bunker system 11, which is a principal object of the present invention.

### Method of Constructing Bunker System 11

The utilization of a dual-core radiation shielding structure 19 greatly facilitates the overall process of constructing bunker system 11. Specifically, with foundation 18 in place, the individual modular blocks 35 that are used to form outer wall 31 are stacked in the required arrangement (i.e., as shown in FIGS. 1-3). As referenced previously, adjacent blocks 35 preferably interlock through the use of complementary projections and recesses to retain outer wall 31 in its proper assembly.

Due to the modular construction of outer wall 31, a customized arrangement of preformed blocks 35 can be used to define certain passageways into which radiation equipment infrastructure (e.g., electrical conduits, coolant piping, exhaust ductwork, and the like) can be arranged. As a result, the various workers involved in the construction of bunker system 11, such as construction engineers, electricians and plumbers, can essentially work in parallel, thereby creating a highly efficient construction process.

For instance, once a first section of the outer wall 31 is assembled using modular blocks 35, electricians can run necessary conduits through designated passageways defined within that section. At the same time, construction workers can assemble additional sections of outer wall 31 in other regions of the room, with further cavities and passageways being defined therein for additional equipment infrastructure. In this capacity, any work-related delays experienced are minimal.

Once outer wall 31 is assembled with all equipment infrastructure properly installed therein, a cast-in-place concrete mixture is preferably deposited within any voids in outer wall 31 to enclose, or solidify, the resultant wall structure. Thereafter, inner wall 33 is preferably formed using cast-in-place construction techniques (i.e., by depositing an unhardened concrete mixture between removable forms). In the present embodiment, inner wall 33 is preferably structurally reinforced (e.g., with steel embedment plates) to support equipment for radiation treatment device 13, which can weigh in the order of several thousand tons. Furthermore, it should be noted that, due to presence of outer wall 31, inner wall 33 can be constructed with a limited thickness, thereby significantly reducing the required curing period.

As referenced briefly above, ceiling structure 20 is constructed in connection with foundation 18 and shielding structure 19 to form an enclosure that surrounds radiation treatment device 13 on all sides and thereby prevents the emission of radiation to the outside environment. Ceiling structure 20 is shaped to include a pair of enlarged access openings 26 through which cyclotron 15 and gantry 17 are delivered into vault 21 and room 23, respectively. Once delivered, access openings 26 are enclosed using an arrangement of removable, concrete roof panels 27.

### FEATURES AND ADVANTAGES OF THE PRESENT INVENTION

As set forth in detail below, the utilization of a dual-core radiation shielding structure provides bunker system 11 with a number of notable advantages over traditional radiation shielding rooms.

As a first advantage, the utilization of preformed, modular blocks 35 significantly accelerates the overall construction timeline by as much as five or more months, which is approximately 35% faster than traditional means. Most notably, modular blocks 35 allow for (i) a reduction in the thickness of cast-in-place concrete used to form radiation shielding structure 19, thereby significantly reducing curing time requirements, and (ii) in parallel, or simultaneous, installation by the various workers involved in building room 11. As a result of this acceleration in construction, a radiation treatment facility can be fully operable and generating income at an earlier date in time. As an added benefit, an accelerated construction process reduces project financing and carrying costs.

As a second advantage, the utilization of preformed, modular blocks 35 provides greater quality control with respect to radiation shielding performance. Specifically, prior its installation, each individual block 35 is manufactured in a controlled environment and, in turn, inspected with respect to its physical, dimensional and weight qualifications. By contrast, the specifications, or attributes, associated with cast-in-place concrete walls have been found to experience notable variances (e.g., with respect to density) due to the inherent inconsistencies in which pour-in-place concrete is manually deposited between forms. As such, by using preformed modular blocks 35 with highly consistent densities, shielding requirements can be more accurately engineered.

As a third advantage, the utilization of preformed, modular blocks 35 affords an architect with greater design conformability. For instance, because the density of modular blocks 35 can be readily varied by using different concrete mixtures, bunker system 11 can be customized for particular applications. In other words, a limited number of modular blocks of higher/increased density concrete can be used to provide bunker system 11 with either (i) greater radiation shielding capabilities in certain critical regions or (ii) the ability to correspondingly reduce the thickness of sections of inner wall 33 in direct alignment therewith in order to accommodate certain architectural limitations (e.g., a unique facility footprint or another similar structural restriction).

As a fourth advantage, the dual-core radiation shielding structure provides bunker system 11 with the ability to reuse or recycle certain components. In particular, the interior surface of a radiation treatment room becomes radioactive over time. However, the depth of radioactivity is typically less than 2 feet. Accordingly, inner wall 33, which is 2 feet in thickness, forms a radiation decontamination layer. Consequently, blocks 35 in outer core 31 would not likely become contaminated and, as a result, could be reused in future bunker systems, which is highly desirable.

As a fifth advantage, the utilization of preformed, modular blocks 35 to construct outer wall 31 enables a portion of radiation shielding structure 19 to be classified as equipment rather than a building. As a result, the treatment facility for system 11 is able to accelerate its depreciation allocation, which creates more immediate tax savings.

The embodiment shown above is intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A bunker system for radiation therapy equipment, the bunker system comprising:
(a) an inner core; and
(b) an outer core that immediately surrounds the inner core;
(c) wherein the inner core and outer core together form a radiation shielding structure.

2. The bunker system as claimed in claim 1 wherein the radiation shielding structure is adapted to surround the radiation therapy equipment.

3. The bunker system as claimed in claim 2 wherein each of the inner core and the outer core is adapted to surround the radiation therapy equipment.

4. The bunker system as claimed in claim 2 wherein the outer core comprises a plurality of preformed modular blocks.

5. The bunker system as claimed in claim 4 wherein each of the plurality of modular blocks is constructed of a radiation shielding material.

6. The bunker system as claimed in claim 5 wherein each of the plurality of modular blocks is constructed of a concrete-based mixture.

7. The bunker system as claimed in claim 5 wherein the plurality of modular blocks together forms a continuous wall-type structure.

8. The bunker system as claimed in claim 7 wherein the plurality of modular blocks is stackable.

9. The bunker system as claimed in claim 8 wherein the plurality of modular blocks is interlocking.

10. The bunker system as claimed in claim 5 wherein the inner core is a cast-in-place concrete structure.

11. The bunker system as claimed in claim 10 wherein the inner core has a thickness that is no greater than approximately 2 feet.

12. The bunker system as claimed in claim 11 wherein the inner core has a thickness of approximately 2 feet.

13. The bunker system as claimed in claim 10 further comprising:
(a) a flooring structure; and
(b) a ceiling structure;
(c) wherein the shielding structure, the flooring structure and the ceiling structure together define a room adapted to receive the radiation therapy equipment.

14. The bunker system as claimed in claim 13 wherein the shielding structure, the flooring structure and the ceiling structure are adapted to completely surround the radiation therapy equipment to prevent emission of radiation from the room.
